(19) United States
(12) Plant Patent Application Publication
(10) Pub. No.: US 2012/0266340 P1
(43) Pub. Date: Oct. 18, 2012

(54) Vuylstekeara celtic sun 'Sunkissed'

(50) ;

(76) Inventor: James William McCully, Hillo, HI (US)

(21) Appl. No.: 13/066,640

(22) Filed: Apr. 18, 2011

(51) Int. Cl. A01H 5/00

(52) U.S. Cl. PLT/311

(57) ABSTRACT

A new variety of orchid plant of the Oncidiinae Intergenerics, Vuylstekeara, named Vuylstekeara Celtic Sun ‘Sunkissed’, distinguished particularly by a clean pleasing arrangement of yellow flowers on a raceme that is desirable in size for the size of the plant and growing quickly to sexual maturity.

### CROSS-REFERENCE TO RELATED APPLICATIONS

Not Applicable

### STATEMENT REGARDING FEDERALLY SPONSORED RESEARCH OR DEVELOPMENT

Not Applicable

### LATIN NAME OF THE GENUS AND GREX OF THE PLANT CLAIMED

The Latin name of the genus and grex of the plant claimed is Vuylstekeara Celtic Sun.

### VARIETY DENOMINATION

The present invention comprises a new and distinct cultivar of Vuylstekeara orchid, and hereinafter referred to by the cultivar name Vuylstekeara Celtic Sun ‘Sunkissed’.

### BACKGROUND OF THE INVENTION

Vuylstekeara is an intergeneric hybrid between three orchid genera, Cochlioda, Miltoniopsis and Odontoglossum within the subtribe Oncidiinae. Cochlioda consists of 13 species, Odontoglossum consists of about 100 species and Miltoniopsis consists of 6 species. The first properly registered hybrid of Vuylstekeara was registered by Lambeau in 1914. Although Charlesworth Ltd. had registered under the name Vuylstekeara a few years previously, the hybrid was not a true Vuylstekeara . Due to the varying percentages of the parent genera and large number of species that can be involved to make up Vuylstekeara and the gene expression in their progeny, Vuylstekeara varies in morphology and size. All exhibit sympodial growth habit.

Vuylstekeara breeding is typically done from sexual methods. Asexual propagation of Vuylstekeara is often done in aseptic tissue culture from apical or axillary shoots.

The new cultivar was discovered within the progeny of a cross made in Hilo, Hi. on Oct. 6, 2003. The Inventor selected Vuylstekeara Celtic Sun Sunkissed' as a single plant from a population of over 200 plants grown in Hilo, Hi. Vuylstekeara Celtic Sun ‘Sunkissed’ was re-flowered for evaluation and submitted by the Inventor to a commercial tissue culture laboratory in Bangkok, Thailand on Dec. 12, 2007 for propagation through aseptic tissue culture technique.

Other seedlings from the same cross that was used to produce Vuylstekeara Celtic Sun ‘Sunkissed’ were sold by the Inventor and may have been commercially available from others. The sibling plants were sold without specific epithet to individuals and other nurseries. To the Inventor's knowledge, the only other named clone of the cross is Vuylstekeara Celtic Sun ‘Maya’ and is owned by the inventor. The inventor has reason to believe that Vuylstekeara Celtic Sun ‘Sunkissed’ is superior to the other seedlings in growth, shape and color. Plants of the grex Vuylstekeara Celtic Sun are sold as plants of the grex without specific epithet.

### BRIEF SUMMARY OF THE INVENTION

The following traits have been repeatedly observed and are determined to be basic characteristics of the new cultivar which in combination distinguish this Vuylstekeara orchid as a new and distinct cultivar:

Flowers of the new cultivar have petals and sepals that are a rich, crystalline yellow. There are varying degrees of a 1×1 cm striated patch of pink on the basal part of the lateral petals that is deepest in color along the parallel venation of the petal. The labellum is pale yellow, almost cream colored, with a yellow patch down the middle distally. The anther cap is gray with a spray paint-like overlay of purple. The callus is yellow surrounded on both sides and irregularly spotted and striated with red. Irregular red spots or striations are sometimes found just below the callus, distally. The column is white at its base on top with yellow on the sides, striated with burgundy red mesially and, once again, white apically. The underside of the column is yellow with a band of burgundy spots basally and is a translucent gray distally at the stigma. The column wings are rectangular-dentate and white with multiple burgundy spots. The flowers are semi-rigid. The natural horizontal spread is 8.4 centimeters (cm); natural vertical spread is 9 cm; dorsal sepal width 2.2 cm; dorsal sepal length 4.3 cm; petal width 2.2 cm; petal length 4.4 cm; lateral sepal width 1.9 cm; lateral sepal length 4.1 cm; labellum width 4.0 cm; labellum length 4.4 cm. The plant usually produces one inflorescence, sometimes two, on the first bloom with and average of 5 flowers per inflorescence.

Inflorescence is a basal raceme, upright or arching, and approximately 33 cm tall. The plant is relatively compact and the size of the inflorescence is appropriate to the overall size of the plant and typical pot size for this cultivar.

There are generally five leaves on the mature pseudobulb, one from the apex and two from each side of the base of the pseudobulb. The apical leaf measures approximately 45 cm long and 3.2 cm wide. The leaves from the base of the pseudobulb measure from 21 to 41 cm long and 3.0 cm wide. Axillary leaves, or bracts, are sometimes present below the leaves at the base of the pseudobulb. When present, there is usually one on either side of the pseudobulb and measure 6 to 16 cm long and 1.4 to 2.2 cm wide. The shape of the leaf is lanceolate acuminate and semi-conduplicate at the base. The texture and substance is very slightly rugose due to parallel vein striation, waxy and thin. The plants of Vuylstekeara Celtic Sun ‘Sunkissed’ grow to maturity and flower in approximately 18 months.

Plants of the new cultivar have not been observed under all possible environmental conditions. The phenotype may vary significantly with variations in the environment such as temperature, light intensity, and day length, without however any change in genotype.

Plants of the new cultivar differ primarily from the plants of the parent cultivars in flower color, shape, rate of growth and speed to maturation. Perhaps the closest commercial comparison to the new cultivar can be made to seedling-derived Vuylstekeara orchids which are genetically heterogeneous, and typically lack uniformity in growth, vigor, plant habit, and flower quality. Since this reference point has inconsistent characteristics, a direct comparison for Vuylstekeara Celtic Sun ‘Sunkissed’ is not available. The new cultivar is a single genotype asexually propagated via tissue culture; thus, its combined horticultural characteristics listed above are uniform and predictable.

### BRIEF DESCRIPTION OF THE DRAWINGS

The drawings show the colors of the new variety as nearly true as possible with an illustration of this type. In the drawings:

FIG. 1 is a view of the entire plant to reveal the growth habit and general characteristics with its flowers, which is typical of this new variety.

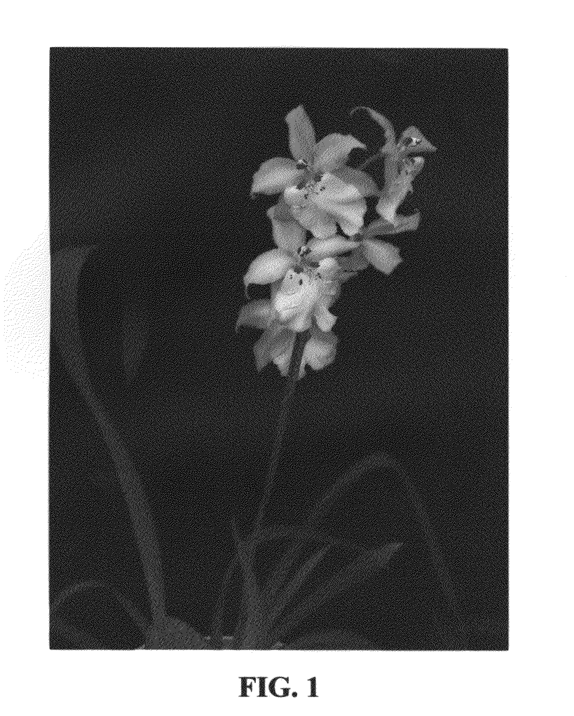

FIG. 2 is a close up view of the flower to show its shape.

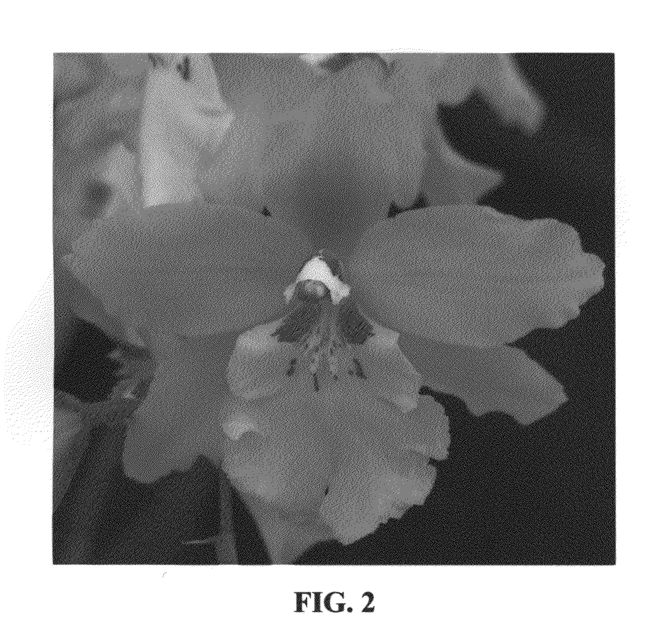

FIG. 3 is a view of the flower showing the patch of pink that can occur on to varying degrees on the lateral petals.

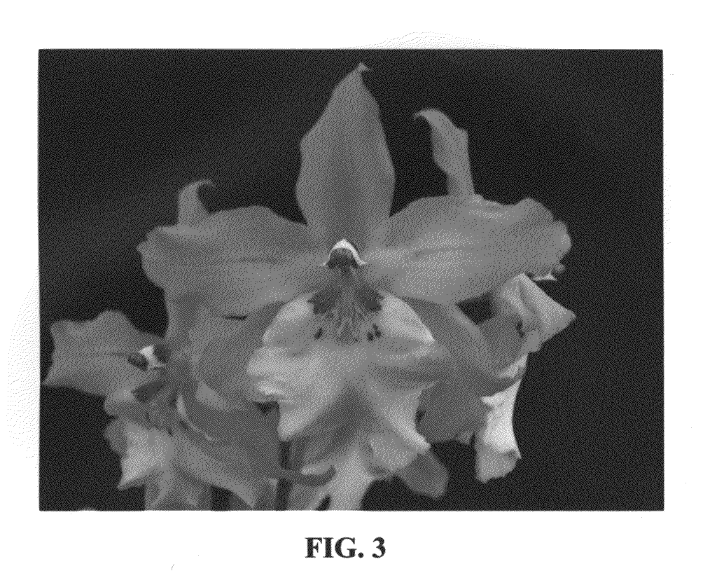

### DETAILED BOTANICAL DESCRIPTION

All color references are measured against the Pantone® Color System. Colors and numerical measurements are approximate as plant growth and development depends on environmental conditions and cultural practices such as light level, temperature, water quality, fertilization formula and rate, among others, without, however, any variance in genotype.

Plants used for this description are 18 months in vivo and grown in square, black plastic pots measuring 3.25″ at the top of the pot and tapering down to 2.5″ at the bottom of the pot with a height of 3.75″, grown in a poly-film covered greenhouse in Hilo, Hi., where day temperatures range from 75 to 85 degrees and night temperatures range from 55 to 65 degrees Fahrenheit. Light levels are approximately 2500 foot candles. This information reflects the annual variations for the area.
Botanical classification: Vuylstekeara Celtic Sun ‘Sunkissed’
Parentage: Seedling selected from the cross of the following:
   Seed Parent.—Miltoniopsis Zorro.
   Pollen Parent.—Odontioda Golden Rialto.
Propagation:
   Type.—Asexual propagation by aseptic tissue culture through axillary shoot initiation.
   Time to initiate and elongate roots in-vivo.—about 45 days.
   Time to produce fully rooted young plant.—about 240 days.
Root description: Velamen covered roots, approximately 1.5 mm in diameter, creamy white in color with green growing tip. The exact shades of white and green vary greatly with minimal changes in environmental conditions.
Plant description: Under appropriate growing conditions, plants of the new cultivar attain a mature size of about 45 cm in height (top of leaf plane) and about 3.2 cm in width. The height from soil level to the top of the inflorescences averages 33 cm.
Foliage description:
   Arrangement.—Sympodial growth habit.
   Quantity of leaves per growth.—Each sympodial growth has shown five leaves, one from the apex of the pseudobulb and four from the axis at the base of the pseudobulb.
   Leaf length.—Apical leaves measure approximately 45 cm in length. The leaves at the base of the pseudobulb may be as short as 21 cm to and as long as 41 cm.
   Leaf width.—The width of the largest leaf measures up to 3.2 cm.
   Leaf shape.—The shape is lanceolate acuminate, entire, apex acute and slightly recurved with a semi-conduplicate base. The leaf margins are smooth, parallel venation apparent, texture and substance of both the upper and lower surfaces of the leaf is minimally rugose, waxy and thin.
   Pseudobulb shape.—The pseudobulb shape is a laterally compressed ovoid.
   Pseudobulb length.—The pseudobulb is approximately 6.5 cm long.
   Pseudobulb width.—The pseudobulb is approximately 4.0cm wide and 0.8 cm thick.
   Foliage color.—Under the described growing conditions, the foliage color on the upper surface of the leaves is similar to Pantone® 574C (green) of the Pantone® Color System. The lower surface of the leaf is a slightly lighter green, Pantone® 574PC. There is no vein color distinction. The pseudobulb color is Pantone® 575PC (green).
Flower/inflorescence description:
   Description.—Inflorescence is an upright or arching raceme, terete, approximately 33 cm in length and 0.3 cm in width on a first bloom plant, typically occurring on the second pseudobulb and grown in square black plastic pots measuring 3.25″ on top, 2.5″ on the bottom with a height of 3.75″. The glabrous peduncles are approximately 22.5 cm in length to the first flower. The pedicels are approximately 1.6 to 2.7 cm in length and 0.3 cm in diameter. Although somewhat pliable when physically bent, they are fairly rigid and strong, supporting each flower sufficiently. The angle of the pedicel is 75-90° giving the flowers proper spacing to display themselves without overcrowding. The texture of the pedicels is fairly smooth and the coloration is light green (Pantone® 377PC). Each raceme averages 5 flowers. Typically, there is one inflorescence per pseudobulb, but two inflorescences per pseudobulb have frequently been observed on this cultivar. The flowers begin approximately two-thirds of the way up the raceme and are spaced around 3 cm apart. The size of the inflorescence is appropriate to the overall size of the plant and typical pot size for this cultivar. The above description is of a first bloom plant in the pot sized as outlined with 2 pseudobulbs. If additional pseudobulbs are grown, each pseudobulb will produce 1-2 racemes with and average of 5 flowers each. The flower buds are conical acute, 0.8 to 1.2 cm in diameter and 2.5 to 3.5 cm in length. The surface is glabrous and the coloration is Pantone® 115C (yellow) overlaid with burgundy in some areas (Pantone® 1807UP). The flowers are bilaterally symmetrical, which is typical of most orchids. Each flower consists of 3 sepals and 3 petals. The lower petal is modified and referred to as the labellum and/or lip. The upper sides of the sepals and petals are yellow in color (Pantone® 115C). The lower sides of the sepals and petals are yellow (Pantone® 115PC) with a light green (Pantone® 382PC) patch on the sepals starting at the base extending down the midrib distally. This patch shows through slightly on the upper surface of the petals as a light green (Pantone®380C). The dorsal sepal is lanceolate, apex apiculate and reflexed for 0.2 cm, margins mostly smooth. The lateral sepals are similar in shape though slightly smaller in length and width and margins slightly undulate. Petals are lanceolate acuminate, apex reflexed for 0.3 to 0.4 cm with somewhat undulate margins. Many times there is a patch of pink (Pantone® 219UP) that appears about 0.5 cm from the base of the lateral petals and extends as a 1×1 cm patch, deepest in color along the parallel venation, but varying degrees of color and shading. Sepals and petals have a sparkly, crystalline texture. The labellum (lip) is a compressed ovoid, bibbed with serrate and undulate margins. The lip lamina is somewhat rectangular shaped at its base. The upper and lower surface of the lip has a satin texture. The lip is a cream to pale yellow (Pantone® 607PC) with a patch of yellow (Pantone® 603PC) down the central lower half of the lip. The throat of the lip is deep yellow (Pantone® 109C) surrounded on both outer sides by red patches (Pantone® 1807C). Occasionally, there are irregular red spots present on the central upper part of the lip Pantone® 1807C). These spots range from 1 to 1.5 mm circular spots to 3×1 mm rectangular bands. The lower surface of the lip is cream to pale yellow (Pantone® 607C), and being somewhat translucent, the coloring and spotting from the upper surface of the lip is somewhat visible upon close inspection. The callus of the labellum begins as a swelling in the throat of the lip which bifurcates into multiple individual lamella that rise up approximately 3.5 mm at the highest point and range from 1 to 5 mm long and equal to or less than 1 mm wide. The texture of the apical surfaces of the calli are smooth, the sides are rigid and smooth. The surface colors of the callus are red (Pantone® 1807C) and yellow (109C).
   Dimensions.—Flower dimensions are: natural horizontal spread is 4.5 cm; natural vertical spread is 6 cm; dorsal sepal width is 1.0 cm; dorsal sepal length is 2.6 cm; lateral sepal width is 0.9 cm; lateral sepal length is 3 cm; petal width is 1.0 cm; petal length is 2.3 cm; labellum width is 3.1 cm; labellum length is 3.3 cm.
   Coloration.—The dominant flower color that appears on the upper and lower surfaces of the sepals and petals is yellow, Pantone® 115C. The patch of pink that can occur on the inner lateral petals to varying degrees is pink, Pantone® 219UP. The dominant color of the upper and lower surfaces of the lip is creamy pale yellow, Pantone® 607PC, upper surface having a patch of darker yellow, Pantone® 603PC, as well of shades of red, Pantone® 1807C, and yellow, Pantone® 109C, on the callus of the lip. Irregular spots and/or bands on the central upper part of the lip below the callus are red, Pantone® 1807C. The upper and side colors of the column at its base is white, Pantone® Standard White, overlaid by striations of red, Pantone® 187UP centrally for approximately 5 mm, and white, Pantone® Standard White, again at the apex. The lower surface of the column is yellow, Pantone® 116C, with a band of irregular spotting horizontally through the middle and red, Pantone® 188C, in color. The column wings are white, Pantone® Standard White, with irregular spotting of burgundy, Pantone® 187U. The stigmatic surface is a translucent gray, Pantone® 7527PC, with a 1 mm burgundy rim, Pantone® 1807UP. The anther cap is light gray, Pantone® 7527PC, with a light to heavy spray paint-like overlay of purple, Pantone® 258UP. The pollinia are completely concealed by the anther cap, and if it is removed the pollinia may be extracted. The pollinia are yellow, Pantone® 124C, but can range in shades of yellow as the plant ages.
Quantity of flowers and time to flower:
   Flower longevity.—The flowers can last longer than 8 weeks on the plant with good environmental conditions. A first bloom plant will produce around 5 flowers depending on environmental conditions. Since this plant has been grown for pot plant purposes, cut flower longevity has not been observed.
   Natural flowering season.—This plant has appeared to be free flowering although the dominant flowering season is winter (January, February, March, April).
Fragrance: No fragrance has been detected by the Inventor.
Reproductive organs: The stamens, style and stigmas are fused into a single short structure called the column, possessing one terminal anther with pollen grains united to pollinia, which are covered by an anther cap. The stigma is located under the column behind the pollinia. The ovary is inferior, with three carpels being present.
   Column.—The column is erect with narrowly lamellate wings on either side of the stigma, 2.5 mm wide by 6 mm long.
   Pollinia.—Two oval masses of pollen are present, about 1.0 mm in diameter and 2.0 mm long.
   Stigma.—The stigma is concave, round, with a high gloss, and sticky. The length of the stigma is 5 mm and the width is 5 mm.
   Ovary.—The ovary is about 2.7 cm long by 0.3 cm in diameter and light green in color (Pantone® 377PC).
   Seed.—Seed production has not been observed.
Disease resistance: Resistance or susceptibility to known pathogens of Vuylstekeara has not been observed on plants grown under commercial production conditions.
General observations: Plants of Vuylstekeara Celtic Sun ‘Sunkissed’ produce a pleasing arrangement of yellow flowers on a raceme that is desirable in size for the size of the plant. The flowers are long lasting and nicely shaped. The plant grows quickly to sexual maturity.

What is claimed is:

1. A new and distinct variety of orchid plant named Vuylstekeara Celtic Sun ‘Sunkissed’, substantially as illustrated and described herein.
